(19) United States
(12) Patent Application Publication
(10) Pub. No.: US 2009/0149876 A1
(43) Pub. Date: Jun. 11, 2009

(54) SURGICAL CLAMP AND METHOD OF CLAMPING AN ORGAN

(76) Inventors: Manoj Patel, Jacksonville, FL (US); David Hoenig, Great Neck, NY (US)

Correspondence Address: MARK YOUNG, P.A., 12086 FORT CAROLINE ROAD, UNIT 202, JACKSONVILLE, FL, 32225, US

(21) Appl. No.: 12/330,508

(22) Filed: Dec. 8, 2008

Related U.S. Application Data
(60) Provisional application No. 61/005,630, filed on Dec. 6, 2007.

(51) Int. Cl. A61B 17/08; A61B 19/00

(52) U.S. Cl. 606/151; 128/898

(57) ABSTRACT

A surgical clamp is provided that is applied through a laparoscopic port and is used for clamping off a portion of an organ. The surgical clamp comprises an elongated flexible bioabsorbable polymer band. The band has a proximal end and a distal end. A bioabsorbable polymer tie secures in place the proximal end of the band to the distal end of the band.

### RELATED APPLICATION

This application is a nonprovisional application and claims the benefit of priority of U.S. provisional application 61/005,630 filed Dec. 6, 2007, the entire contents of which are incorporated herein by this reference and made a part hereof.

### FIELD OF THE INVENTION

The present invention relates to surgical clamps, and more particularly to laparoscopically applied bioabsorbable surgical clamps.

### BACKGROUND OF THE INVENTION

It is known to use surgical clamps to clamp off portions of tubular bodily vessels or organs, such as blood vessels, bowels, ducts, urethra and the like. Conventional clamps for tubular organ structures include scissor-type clamps. However, scissor-type clamps are not suitable for adapting to organs of different sizes and shapes. Thus, it is known to use loop-type surgical clamps to clamp off portions of non-tubular organs, such as the liver, kidney, and the like. Conventional clamps loop-type clamps described in U.S. Pat. No. 5,203,786 (Vernick) and U.S. Pat. No. 5,304,188 (Marogil).

It is also known to apply a scissor-type clamp through a laparoscopic port, such as trocar, using a laparoscopic applier. In laparoscopic procedures the inside diameter of the trocar limits the size of the clamp and applier that may be used. Laparoscopically applied scissor-type clamps are described in U.S. Pat. No. 5,368,600 (Failla et al.) and U.S. Pat. No. 5,496,333 (Sackier et al.). However, there is a need for a loop-type clamp that is applied through a trocar during laparoscopic surgery.

In certain situations, it may be desirable to leave the clamp inside the patient's body so that the organ remains clamped off after surgery. It is also desirable to avoid having to perform an additional surgical procedure to remove the clamp. Thus, there is a need for a loop-type clamp that can be left in patient's body.

### SUMMARY OF THE INVENTION

According to one embodiment, a surgical clamp is provided that is applied through a laparoscopic port and is used for clamping off a portion of an organ. The surgical clamp comprises an elongated flexible bioabsorbable polymer band. The band has a proximal end and a distal end. A bioabsorbable polymer tie secures in place the proximal end of the band to the distal end of the band.

According to one embodiment, a method of clamping off a portion of an organ in a surgical patient comprises inserting a bioabsorbable band of a surgical clamp through a laparoscopic port. The distal end of the band is coupled to a proximal end of the band with a bioabsorbable tie. The band is elongatedly folded such that a portion of the inner surface of the distal end of the band abuts with a portion of the inner surface of the proximal end of the band forward of the tie. The forward portion of the band is expanded into a loop after passing through the laparoscopic port. The loop portion of the band is encircled around a portion of the organ. The band is tightened around the organ. These and other features of this invention are described in, or are apparent from, the following detailed description of various exemplary embodiments of this invention.

### BRIEF DESCRIPTION OF THE DRAWINGS

Exemplary embodiments of this invention will be described with reference to the accompanying figures.

FIG. 1 is a laparoscopic surgical clamp according to an exemplary embodiment.

FIGS. 2-4 are laparoscopic surgical clamps according various exemplary embodiments in which one end of the band is inserted through the tie.

FIGS. 5-6 are laparoscopic surgical clamps according to exemplary embodiments in which both ends of the band are inserted through the tie.

FIG. 7 is a handle for laparoscopically applying the surgical clamp according to an exemplary embodiment.

### DETAILED DESCRIPTION OF THE EXEMPLARY EMBODIMENTS

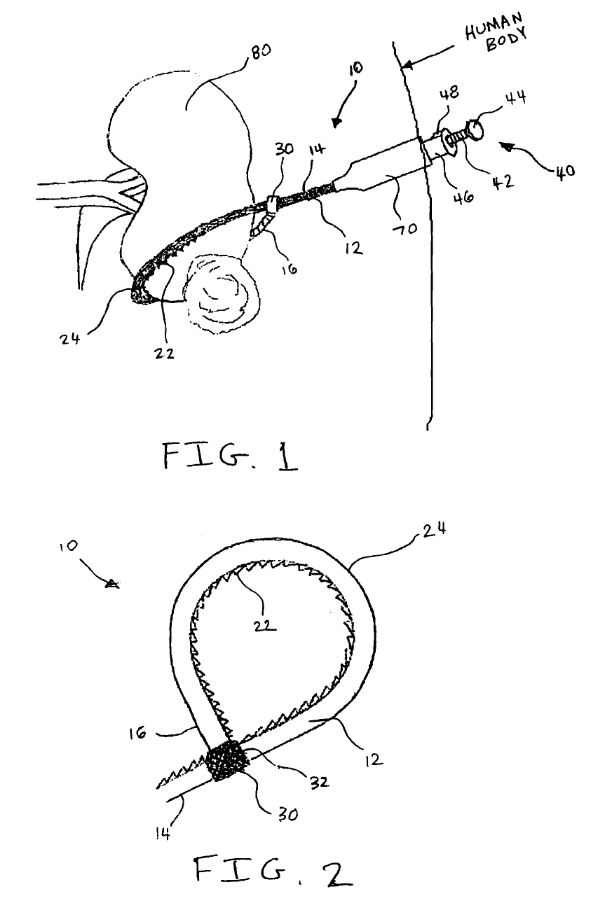

FIG. 1 is a laparoscopic surgical clamp according to an exemplary embodiment. Referring to FIG. 1, a surgical clamp 10 includes a flexible elongated band 12 having a proximal end 14 and a distal end 16. The band 12 is preferably composed of a material that is biodegradable and bioabsorbable in the body during clinical applications. For example, the band 12 may be composed of a bioabsorbable and biodegradable polymer, such as polyglactin, polydioxanone (PDQ), polycaprolactone (peL), polylactic acid (PLA), polyglycolic acid (PGA), poly(lactic-co-glycolic acid) (PLGA), polyhydroxybutyrate (PHB), or the like.

A tie 30 secures together the proximal and distal ends 14, 16 of the band 12 to form a loop 24. The loop portion 24 may be encircled around an organ 80 that is to be clamped, such as a kidney, a liver, etc. The tie 30 is moveable to constrict the loop portion 24 around the organ 80, but is lockable so that the loop portion 24 can be secured in place around the organ 80. The tie 30 is preferably composed of a material that is biodegradable and bioabsorbable in the body during clinical applications. For example, the tie 30 may be composed of a bioabsorbable and biodegradable polymer, such as polyglactin, polydioxanone (POD), polycaprolactone (peL), polylactic acid (PLA), polyglycolic acid (PGA), poly(lactic-co-glycolic acid) (PLGA), polyhydroxybutyrate (PHB), or the like.

The loop portion 24 is foldable so that it may be inserted through a laparoscopic port 70, such as a trocar. Specifically, the loop portion 24 is elongatedly folded such that the inner surface of a portion of the proximal end 14 of the band 12 abuts the inner surface of a portion of the distal end 16 of the band 12. Preferably, the folded loop portion 24 has an overall thickness and width sized such that it may be inserted into an 18 mm trocar port. More preferably, the folded loop portion 24 has an overall thickness and width sized such that it may be inserted into a 12 mm trocar port.

The band 12 includes a plurality of raised projections 22 that protrude from the one of the inner or outer surfaces 18, 20 of the band 12. When the raised projections 22 are formed on the inner organ contacting surface 18 of the band 12, the raised projects can provide an enhanced gripping surface for holding the clamp 10 around the organ 80 without slipping. The raised projections 22 may also be utilized in conjunction with the tie 30 to secure the clamp 10 in place around the organ 80. For example, the raised projections 22 may comprise bumps, studs, knobs, teeth, ribs, ridges, or the like.

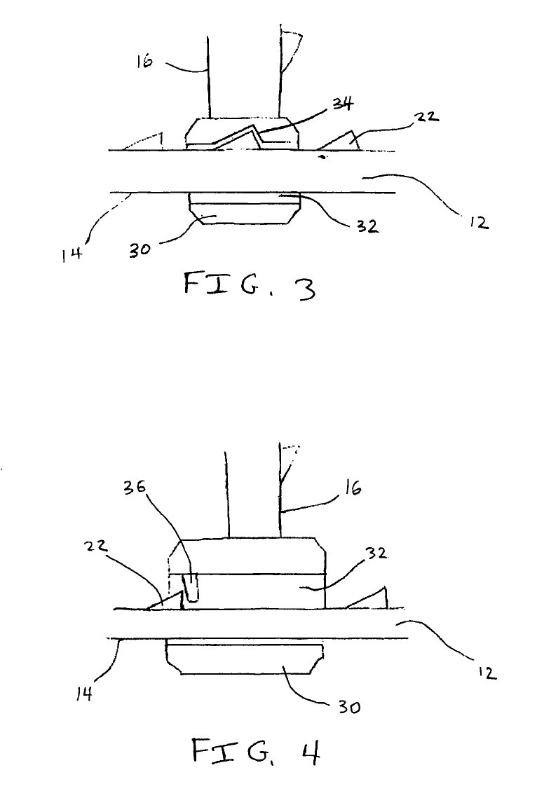

FIGS. 2-4 illustrate laparoscopic surgical clamps according various exemplary embodiments in which one end of the band is inserted through the tie.

Referring to FIGS. 2-4, the tie 30 is fixedly attached to the distal end 16 of the band 12. The tie 30 includes an aperture 32 through which the proximal end 14 of the band 12 is inserted to form the loop portion 24. The proximal end 14 of the band 12 is slidable through the aperture 32.

Raised projections 22 are formed on a surface of the band 12 and are utilized as ratchet teeth to bias against reverse movement of the band 12 through the tie 30. The raised projections 22 are shaped to allow the band 12 to be translated proximally through the aperture 32 to reduce the size of the loop portion 24 and tighten the clamp 10 around the organ 80, while resisting distal movement of the band 12 that would enlarge the size of the loop portion 24 and loosen the clamp 10.

The tie 30 may include a means for disengaging the raised projections 22 to permit distal movement of the band 12 through the tie 30, thereby allowing the clamp 10 to be loosened.

In FIG. 2, the band 12 is translated proximally through the aperture 32 of the tie 30 so that the entire raised projection 22 is extended through the tie 30. If the band 12 is pulled in the distal direction, the distal surface of the raised projection 22 engages against the proximal surface of the tie 30, which prevents distal movement of the band 12 through the aperture 32.

To permit distal movement of the band 12, the raised projections 22 may be preset to slip when a predetermined amount of distal force is applied by varying the incline of the engaging surfaces of the raised projections 22 and the tie 30.

In FIG. 3, one or more recesses or grooves 34 are formed into the inner surface of the tie 30 within the aperture 32. As the band 12 is translated proximally through the aperture 32, the raised projections 22 slide into the recesses 34. When the band 12 is pulled in the distal direction, the distal surfaces of the raised projections 22 engage against the proximal surfaces of the recesses 34 to prevent distal movement of the band 12 through the aperture 32.

To permit distal movement of the band 12, the raised projections 22 may be preset to slip when a predetermined amount of distal force is applied by varying the incline of the engaging surfaces of the raised projections 22 and the recesses 34.

In FIG. 4, a pawl 36 protrudes from an inner surface of the tie 30 and extends into the aperture 32. The band 12 is translated proximally through the aperture 32 so that the entire raised projection 22 is extended past the pawl 36. When the band 12 is pulled in the distal direction, the distal surface of the raised projection 22 engages against the proximal surface of the pawl 36 to prevent distal movement of the band 12 through the aperture 32.

To permit distal movement of the band 12, the tie 30 may include a trigger that disengages the pawl 36 from the raised projections 22 or the raised projections 22 may be preset to slip when a predetermined amount of distal force is applied by varying the incline of the engaging surfaces of the raised projections 22 and the pawl 36.

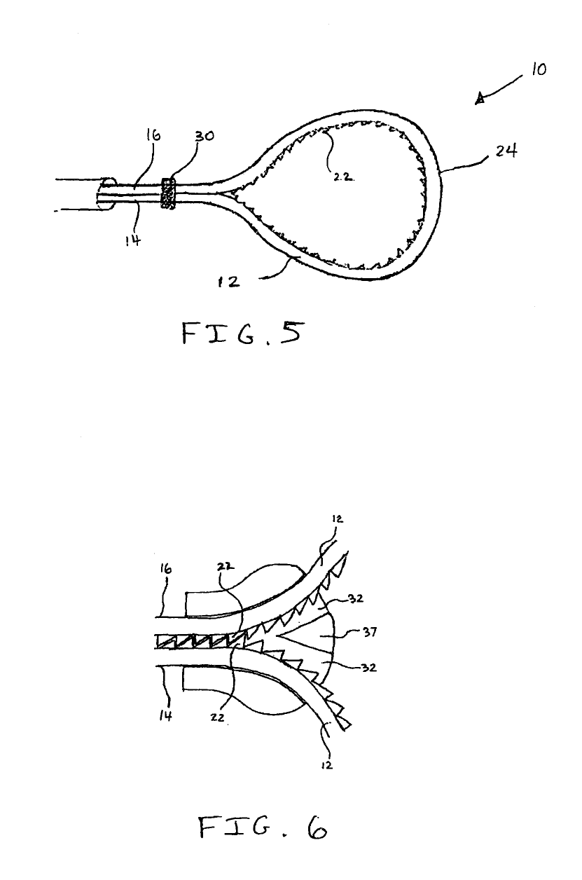

FIGS. 5-6 illustrate laparoscopic surgical clamps according to exemplary embodiments in which both ends of the band are inserted through the tie.

Referring to FIG. 5, the tie 30 includes an aperture 32 through which both the proximal and distal ends 14, 16 of the band 12 are inserted to form the loop portion 24.

Both the distal and proximal ends 14, 16 of the band 12 are slidable through the tie 30. Alternatively, the tie 30 may be fixedly connected at a position along a length one of the distal or proximal ends 14, 16 of the band 12, so that only one of the distal or proximal ends 14, 16 is slidable through the tie 32.

Raised projections 22 are formed on the inner organ contacting surface 18 of the band 12. When the proximal and distal ends 14, 16 of the band 12 are translated proximally through the aperture 32 to reduce the size of the loop portion 24, the inner surfaces 18 of the proximal and distal ends 14, 16 are placed in abutting engagement with each other. The raised projections 22 on the inner surface 18 of the proximal end 14 interlock with the raised projections 22 on the inner surface 18 of the distal end 16, thereby locking the proximal and distal ends 14, 16 of the band 12 together, such as in a zip fastener. The interlocking raised projections 22 have a profile that facilitates interlocking of the raised projections 22 and prevents expansion of the looped portion 24 once the raised projections 22 are engaged.

Referring to FIG. 6, the tie 30 may optionally include a means for disengaging the raised projections 22 so that the band 12 may be moved distally through the aperture 32 to enlarge the size of the loop portion 24. For example, the aperture 32 of the tie 30 may be a Y-shaped channel. When the interlocked proximal and distal ends 14, 16 of the band 12 are moved in the distally through the V-shaped channel 32 of the tie 30, a V-shaped portion 37 of the tie 30 forces the interlocked raised projections 22 to disengage. The distal and proximal ends 14, 16 are separated from each other, which expands the looped portion 24.

The tie 30 may be lockable by an additional locking means. For example, raised projections 22 may also be formed on the outer surface 20 of the band 12, which are utilized to lock the tie 30 in place in a similar fashion as described above with respect to FIGS. 2-4.

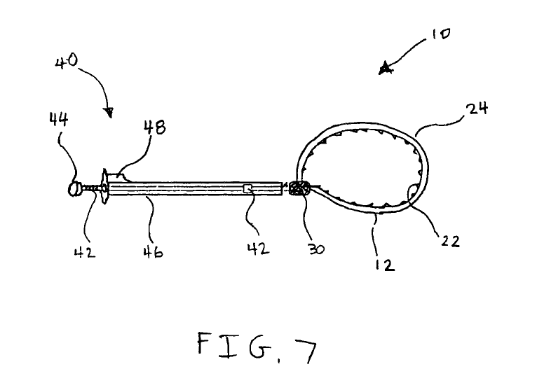

FIG. 7 illustrates a handle for laparoscopically applying the surgical clamp according to an exemplary embodiment.

Referring to FIG. 7, the surgical clamp 10 includes a handle 40 that is adapted to guide the clamp 10 through a laparoscopic port 70, such as a trocar, to the organ 80. The handle 40 is also adapted to tighten or loosen the looped portion 24 of the clamp 10 around the organ 80.

The handle 40 includes an elongated shaft 42 having a grip portion 44 at the proximal end. The distal end of the shaft 42 is connected to the band 12 of clamp 10 at one or both of the distal and proximal ends 14, 16. For example, where the distal end of the band is inserted through the tie as in FIGS. 2-4, the elongated

The shaft 42 is concentrically disposed within an outer housing 46. At least a portion outer housing 46 is internally threaded and at least a portion of the outer surface of the shaft 42 is threaded. Rotation of the shaft 42 along its longitudinal axis within the outer housing 46 causes the shaft 42 to translate in the proximal or distal direction through the outer housing 46. The translation of the shaft 42 exerts a proximal or distal force on the band 12 to move through the tie 30, thereby tightening or loosening the looped portion 24 around the organ 80.

Preferably, the outer housing 46 has a diameter sized such that it may be inserted into an 18 mm trocar port. More preferably, the outer housing 46 has a diameter sized such that it may be inserted into a 12 mm trocar port.

The shaft 42 and the band 12 are secured together by a latch 42. The grip portion 46 of the shaft 42 includes a release member 48 for releasing the latch 42. When the latch 42 is released, the shaft 42 is detached from the band 12. Thus, the clamp 10 may remain attached to the organ 80 inside the surgical patient's body while the handle 40 is removed.

Now that exemplary embodiments of the present invention have been shown and described in detail, various modifications and improvements thereon will become readily apparent to those skilled in the art. Accordingly, the spirit and scope of the present invention is to be construed broadly and limited only by the appended claims, and not by the foregoing specification.

What is claimed is:

1. A surgical clamp applied through a laparoscopic port for clamping off a portion of an organ, comprising: an elongated flexible bioabsorbable polymer band having a proximal end and a distal end; and a bioabsorbable polymer tie that secures in place the proximal end of the band to the distal end of the band.

2. The surgical clamp of claim 1, wherein the bioabsorbable polymer comprises at least one of polyglactin, polydioxanone, polycaprolactone, polylactic acid, polyglycolic acid, poly(lactic-co-glycolic acid), and polyhydroxybutyrate.

3. The surgical clamp of claim 1, further comprising: a detachable handle being operable to insert the band through the laparoscopic port and to tighten the band around the organ.

4. The surgical clamp of claim 3, wherein the handle comprises: an outer housing, at least a portion of the outer housing being internally threaded; and an elongated shaft concentrically disposed with the outer housing, at least a portion of the outer surface of the shaft being threaded, wherein the shaft is rotated along its longitudinal axis in relation to the outer housing to translate a portion of the band through the tie.

5. The surgical clamp of claim 3, further comprising:
a latch securing the handle to the band, and
a release member for detaching the handle from the band, [symbol]
distal end of the band abuts with a portion of the inner surface of the proximal end of the band forward of the tie;
expanding the forward portion of the band into a loop after passing through the laparoscopic port;
encircling the loop portion of the band around a portion of the organ; and
tightening the band around the organ.

14. The method of claim 13, wherein a handle is used to guide the band through the laparascopic port and to encircle the loop portion around the organ, the method further comprising:
disengaging the handle from the band, thereby leaving the band secured in place around the organ inside the patient's body.

15. The method of claim 14, wherein tightening the band around the organ includes rotating the handle along its longitudinal axis to translate a portion of the band through the tie.

16. The method of claim 14, wherein the handle includes a latch securing the handle to the band and a release member for detaching the handle from the band, and wherein disengaging a handle of the clamp from the band includes depressing the release member to disengage the latch.

17. The method of claim 13, wherein the band includes a plurality of raised members formed on a surface of the band, and wherein after the band is tightened around the organ, the distal surface of at least one of the raised projections engages against the tie to bias against distal movement of the band.

18. The method of claim 13, wherein the band includes a plurality of raised projections formed on an inner surface of the band, and wherein when the band is tightened around the organ, the raised projections on one portion of the inner surface of the band are interlocked with the raised portions on an abutting portion of the inner surface the band to secure the abutting portions of the band together.

19. The method of claim 13, wherein the laparoscopic port has a diameter of about 18 mm or less.

20. The method of claim 13, wherein the laparoscopic port has a diameter of about 12 mm or less.
